(19) United States
(12) Patent Application Publication
(10) Pub. No.: US 2023/0194597 A1
(43) Pub. Date: Jun. 22, 2023

(54) TESTING APPARATUS AND METHOD

(71) Applicant: Seer Medical Pty Ltd, Melbourne, Victoria (AU)

(72) Inventor: Kyle Damon SLATER, Melbourne, Victoria (AU)

(73) Assignee: Seer Medical Pty Ltd, Melbourne, Victoria (AU)

(21) Appl. No.: 17/997,176

(22) PCT Filed: Apr. 28, 2021
(86) PCT No.: PCT/AU2021/050381

§ 371 Date: Oct. 26, 2022

(30) Foreign Application Priority Data
Apr. 28, 2020 (AU) 2020901338

(51) Int. Cl. G01R 31/28; G01R 1/067; G06K 7/14

(52) U.S. Cl. CPC G01R 31/2896; G01R 1/06722; G01R 31/2884; G06K 7/1413

(57) ABSTRACT

A capacitive test apparatus for testing an electronic device contained within a package, the apparatus comprising: a plurality of conductive pads in electrical communication with the processor, wherein the plurality of conductive pads are configured to align with electrodes of the electronic device during testing and capacitively couple electrical stimuli to the electrodes via the package; processing circuitry configured to: apply the electrical stimuli to the plurality of conductive pads; receive one or more response signals induced by the electrical stimuli coupled through the package; and determine one or more electrical characteristics of the electronic device based on the one or more response signals.

### TECHNICAL FIELD

This application relates to methods and apparatus for testing electronic devices, in particular electronic device in packaging.

### BACKGROUND

During or after manufacture, electronic devices and subassemblies are typically quality tested using a device commonly referred to as a “test jig”. The purpose of a test jig is to identify and correct manufacturing defects as quickly as possible. An electrical test jig for a printed circuit board (PCB) typically comprises an electrical test circuit having a plurality of pogo pins (spring-loaded pins) for connection to the test circuit. The test jig is brought into proximity of test points on a PCB under test, such that the pogo pins make electrical connection with the test points. These pogo pins may provide power to the PCB and send and receive signals to test that the PCB operates as expected.

For a medical device, particularly one which might be attached to or implanted under the skin of a patient, it is desirable to test operation before the medical device is attached or implanted to avoid consequences of implanting or attaching a faulty medical device.

It is also desirable to perform such tests before removing the device from sterile packaging within which they are typically transported from a manufacturer. This reduces the risk of contamination prior to attachment or implantation. Additionally, if an issue is identified, the unopened sterile package can be returned to the manufacturer without having been tampered with.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to a first aspect of the disclosure, there is provided a capacitive test apparatus for testing an electronic device contained within a package, the apparatus comprising: a plurality of conductive pads in electrical communication with the processor, wherein the plurality of conductive pads are configured to align with electrodes of the electronic device during testing and capacitively couple electrical stimuli to the electrodes through the package; processing circuitry configured to: apply the electrical stimuli to the plurality of conductive pads; receive one or more response signals induced by the electrical stimuli coupled through the package; and determine one or more electrical characteristics of the electronic device based on the one or more response signals.

The package may be at least partially comprised of a dielectric material.

The one or more characteristics may comprise a continuity of an electrical connection in the electronic device; and/or an impedance of the electrical connection in the electronic device.

The processing circuitry may be further configured to: determine that the electronic device is operating properly based on the determining of the one or more characteristics.

The processing circuitry may comprise a signal generator configured to generate the electrical stimuli.

The processing circuitry may comprise one or more voltage buffers coupled to the one of the plurality of conductive pads. The one or more voltage buffers may each comprise a band-stop filter having a stop-band of between about 50 Hz and about 60 Hz.

The processing circuitry may comprise a bandpass filter configured to filter one or more of the one or more response signals. The bandpass filter may have a passband which spans the frequency of the electrical stimuli.

The processing circuitry may comprise an analogue to digital converter configured to convert the one or more response signals into the digital domain.

The processing circuitry may comprise a rectifier configured to rectify the one or more response signals.

The package may be at least partially substantially transparent.

The apparatus may further comprise a camera or similar image capture device configured to capture an image of the electronic device and the package. The processor may be configured to process the image to determine a condition of the electronic device. The processor in conjunction with the camera, a scanner or image capture device may be configured to read a barcode on the package captured in the image.

The apparatus may further comprise a display for displaying the one or more electrical characteristics of the electronic device.

The apparatus may further comprise one or more engagement elements configured to engage with features of the package so as to align the conductive pads with the respective electrodes of the electronic device during testing.

The apparatus may further comprise one or more direct test electrodes configured to directly connect to a respective one or more contacts of the electronic device exposed through the package. The one or more direct test electrodes may each comprise a pogo pin configured to make direct connection with the one or more contacts during testing. The one or more direct test electrodes may directly connect to the one or more contacts via a self-healing membrane.

The package may comprise a plurality of contact regions each configured to receive a respective one of the plurality of test contacts to hold the respective one of the plurality of test contacts in place relative to the package and the plurality of conductive pads during testing.

The package may be stackable.

According to a another aspect of the disclosure, there is provided a method for testing an electronic device housed within a package, the method comprising: locating a plurality of conductive pads in proximity of respective electrodes of the electronic device; capacitively coupling electrical stimuli from the conductive pads to the electrodes through the package; receiving one or more response signals induced by the electrical stimuli coupled through the package; and determining one or more electrical characteristics of the electronic device based on the one or more response signals.

The package may be at least partially comprised of a dielectric material.

The one or more characteristics may comprise one or more of a continuity of an electrical connection in the electronic device; and an impedance of the electrical connection in the electronic device.

The method may further comprise determining that the electronic device is operating properly based on the determining of the one or more characteristics.

The method may further comprise generating the electrical stimuli.

The method may further comprise buffering the one or more response signals prior to determining the one or more electrical characteristics.

The method may further comprise applying a band-stop filter to the one or more response signals. The band-stop filter may have a stopband of between about 50 Hz and about 60 Hz.

The method may further comprise applying a bandpass filter to one or more of the one or more response signals The bandpass filter may have a passband which spans the frequency of the electrical stimuli.

The method may further comprise converting the one or more response signals into the digital domain.

The method may further comprise rectifying the one or more response signals.

The method may further comprise: capturing an image of the electronic device and the package; and processing the image to determine a condition of the electronic device and/or to read a barcode on the package captured in the image.

The method may further comprise displaying the one or more electrical characteristics of the electronic device on a display.

Locating the plurality of conductive pads in proximity of respective electrodes of the electronic device may comprise engaging one or more engagement elements associated with the conductive pads with features of the package so as to align the conductive pads with the respective electrodes of the electronic device during testing.

The method may further comprise directly connecting one or more direct test electrodes to a respective one or more contacts of the electronic device exposed through the package. The one or more direct test electrodes may each comprise a pogo pin configured to make direct connection with the one or more contacts during testing. The one or more direct test electrodes may directly connect to the one or more contacts via a self-healing membrane. The method may further comprise locating each of the plurality of test contacts a respective one of a plurality of contact regions provided in the package. The plurality of contact regions may be configured to hold the respective one of the plurality of test contacts in place relative to the package and the plurality of conductive pads during testing.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of nonlimiting example with reference to the accompanying drawings, in which:

FIG. 1 is a plan view of an electronic device in packaging according to an embodiment of the present disclosure;

FIG. 2 is a side view of a the electronic device and packaging shown in FIG. 1 being tested by a test apparatus according to an embodiment of the present disclosure;

FIG. 3 is a block diagram of a test unit of the test apparatus shown in FIG. 2;

FIG. 4 is a schematic diagram of a measurement circuit of the test unit shown in FIG. 3; and

FIG. 5 is a flow diagram depicting a process performed by the test apparatus shown in FIG. 2.

### DESCRIPTION OF EMBODIMENTS

Embodiments of the present disclosure address or at least alleviate some of the problems associated with prior art testing techniques. In particular, embodiments of the present disclosure enable capacitive coupling of electrical stimuli through the packaging to electrical contacts of a packaged electrical device via the package. The packaging is preferably manufactured from dielectric material to improve the coupling of electrical stimuli to and from electrical contacts of the packaged electrical device.

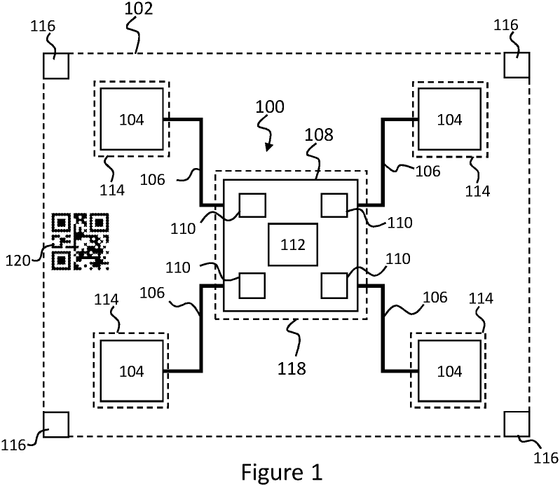

FIG. 1 shows a simplified plan view of an electronic device 100 and device package 102 according to an embodiment of the present disclosure. The electronic device 100 comprises a plurality of electrical contacts 104 electrically coupled via a plurality of wires 106 to a PCB 108. The PCB 108 comprises one or more electrical contacts 110 which may be electrically coupled, via the plurality of wires 106 and via optional circuitry 112 mounted on the PCB 108, to the electrical contacts 104. The primary function of the electrical contacts 110 may be for a function other than for testing the device 100.

The electronic device 102 may be fully or partially encapsulated in the package 102 to protect the electronic device 102 from ingress of dirt, bacteria and/or any other foreign matter. Before use of the electronic device 102 for its intended purpose, the package 102 may be removed. In some embodiments, the electronic device 102, the package 102 or both may be fully or partially sanitised or sterilised prior to the electronic device being placed in the package 102.

Each of the plurality of electrical contacts 104 may be held in place in the package 102 in a respective compartment 114 provided in the package 102. Each compartment 114 may, for example, be formed as a depression in the package 102. Clips or other engagement features (not shown) may be provided in the compartments 114 to further secure the electrical contacts 104 in place in the package.

The exterior of the package 102 may be provided with engagement features 116 configured for engagement with corresponding features of a test assembly 200 so as to hold the package 102 in position relative to the test assembly, as described below in further detail. The package 102 may be stackable such that multiple packages 102 can be stacked on top of one another to save space during transit.

The package 102 may be provided with a test window 118 configured to allow direct connection to electrical contacts 110. In some embodiments, the test window 118 may comprise a self-healing membrane (not shown). The self-healing membrane may comprise a pore-filling hydrogel. The self-healing membrane may provide some protection from dirt and/or bacteria ingress into the package 102 whilst enabling test pads to pierce the membrane and electrical connect to the electrical contacts 110. Alternatively, the test window 118 may comprise an adhesive sticker or similar.

In use, an operator or machine may peel back the sticker to reveal the electrical contacts 110 shortly before a test is undertaken. In alternative embodiments, the test window 118 is not provided. In which case the test apparatus 200 described below may capacitively couple signals to and from the electrical contacts 110 via the package 102 in a similar manner to that described below for the electrical contacts 104.

The package 102 may comprise a dielectric material, for example a material suitable for forming the dielectric of a capacitor. In some embodiments, the package 102 may comprise a plastic. Example plastics having dielectric properties include high density polyethylene (HDPE), low density polyethylene (LDPE), polyethylene terephthalate (PET), polypropylene, polystyrene and polyvinyl chloride (PVC).

Preferably the package 102 is made from a substantially transparent material to allow visual inspection of the electronic device 100 inside the package 102 by a human or machine. A barcode 120 or other identification mark may be printed or otherwise fixed to the outside of the package 102, the electronic device 100, or both to allow the package to be identified. The barcode 120 may be one-dimensional or multidimensional (e.g. a Quick Response (QR) (RTM) code). Additionally or alternatively, the package 102 may be provided with a short range wireless transmitter/receiver tag, such as an RFID tag with a unique identifier allowing the package 102 to be identified using a scanner associated with the test assembly 200. The wireless tag or an additional wireless tag may also be provided on the electronic device 100 instead of the package 102, particularly where the package 102 is transparent. Additionally or alternatively, the package 102 and/or the device 100 may be provided with one or more coloured elements to enable the device 100 to be identified by image processing of images captured of the package 102 and/or device 100.

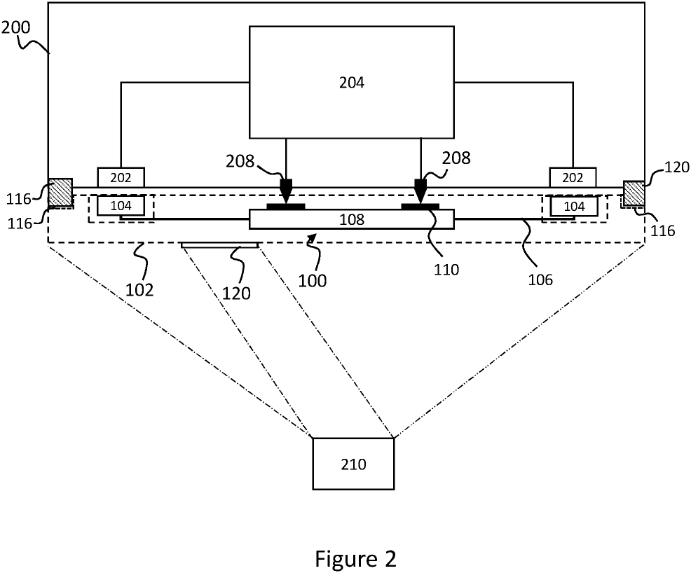

FIG. 2 is a side view of the electronic device 100 and package 102 under test by a test apparatus 200 (or test jig) according to embodiments of the present disclosure. The test apparatus 200 comprises a plurality of conductive test pads 202 electrically coupled to a test unit 204. The apparatus 200 further comprises a plurality of engagement features 206 configured to engage with the engagement features 116 of the package 102 under test, so as to align the conductive test pads 202 of the test apparatus 200 with the electrical contacts 104 of the electrical device 102. The apparatus 200 may further comprise a mechanism (not shown) to secure the package 120 into close proximity of the test apparatus 200 for testing. Such mechanisms are well known from conventional test jigs and so will not be described in any more detail here.

In addition to the conductive test pads 202, the apparatus 200 may comprise one or more test probes 208 configured to make physical contact with the electrical contacts 110 on the PCB 108 of the electronic device 100. The test probes may comprise pogo pins or other similar spring contacts. Where the test window 118 is provided with a self-healing membrane or the like, the test probes 208 may be formed as a spike to as to pierce the test window 118 to achieve electrical connection with the electrical contacts 110 whilst minimizing damage to the self-healing membrane to enable it to heal once the test probes 208 are withdrawn. In alternative embodiments, where the test window 118 is not provided, the test probes 208 may terminate in a pad to form a capacitive coupling through the package 102 between the test probes 208 and the electrical contacts 110, similar to that formed with the electrical contacts 104. A mixture of capacitive coupling and direct physical connection may be provided in some embodiments, with some of the test probes 208 operating as capacitive couplers and others operating as conventional electrical connections. In any case, the one or more test probes 208 may be spring biased so as to ensure either a good physical connection or a good capacitive coupling with the electrical contacts 110 when the device 100 is secured to the test apparatus 200. One or more of the conductive pads 202 and the test probes 208 may be mounted on a PCB (not shown) incorporated into the test apparatus 200.

The test apparatus 200 may further comprise a camera 210, scanner or equivalent input device for obtaining an image and other data from the surface of the package 102 and the electronic device 100 (where the package 102 is formed of transparent material). The camera 210 may capture images of the package 102 (such as the barcode 120) and electronic device 100 before the apparatus 200 is engaged with the package 102. Additionally or alternatively, the camera 210 may capture images during testing of the device 102. Images and other data captured by the camera 210 may be transmitted to the test unit 204 for processing. Captured images may then be analysed by the processor 204 to identify any visible faults or manufacturing defaults in the electronic device 102.

During operation, as the test apparatus 200 is brought into proximity of the package 102 and aligned such that the engagement features 206 engage with one another, the conductive pads 203 are brought into proximity with the electrical contacts 104, sandwiching the package 102 therebetween. In doing so, a capacitor is formed between each of the conductive pads 202, the electrical contacts 104 and the package 102. In this arrangement, signals generated by the processor 104 can therefore be capacitively coupled to the electrical device 100 through the package 102.

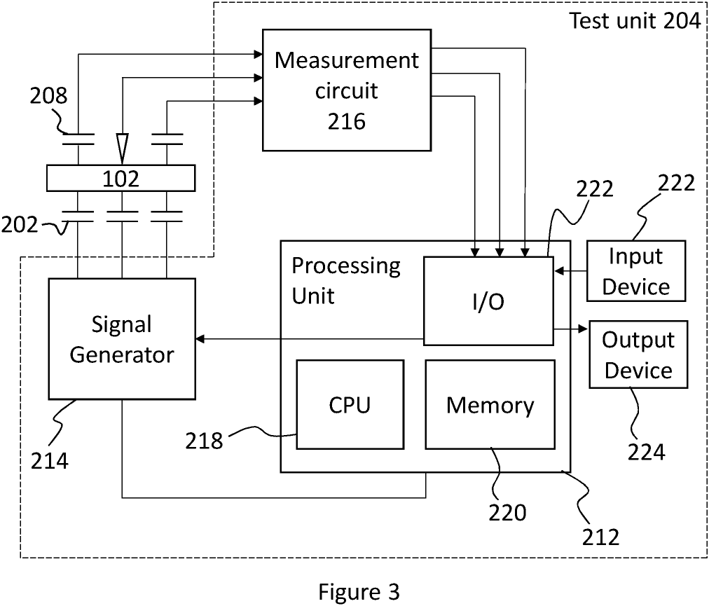

FIG. 3 is a detailed block diagram of the test unit 204 of the test apparatus 200 according to embodiments of the present disclosure. The test unit 204 comprises the a processing unit 212, a signal generator 214 and a measurement circuit 216. The processing unit 212 may comprise a central processing unit (CPU) 218, memory 220, and an input/output (I/O) bus 222 communicatively coupled with one or more of the CPU 218 and memory 220.

In the embodiment shown, the signal generator 214 comprises a plurality of outputs, each coupled to a respective one of the test pads 202. In other embodiments, a multiplexer (not shown) may be provided at the output of the signal generator to selectively couple the signal generator to one of the test pads 202. Equally, a multiplexer may be provided in some embodiments between the output of the test electrodes 208 and the measurement circuit for selective coupling of the measurement circuit 216 to one or more of the test electrodes 208. The signal generator 214 is coupled to one or more of the test pads 202 and operable to deliver electrical stimuli to the test pads 202 based on signals received from the processing unit 212. To this end, the signal generator 214 and the processing unit 212 are communicatively coupled such that information can be transferred therebetween. The signal generator 214 may be external to or integrated within the processing unit 212.

Under control of the processing unit 212, the signal generator 214 may be configured to generate and apply oscillating stimuli to each of the test pads 202 of the test apparatus 200. In some embodiments, the stimulus has a frequency of between 5 kHz and 15 kHz, for example 10 kHz. The stimulus may have an amplitude of between 1.8 V and 5 V, for example between 3 V to 3.5 V, for example 3.3 V. The required frequency and voltage of the stimulus may depend on, amongst other things, the dielectric properties of the package 102, the quality of coupling between the test pads 202 and the electrical contacts 104 of the electrical device, and the sensitivity of the measurement circuit 216. Optionally, an ultra-high impedance buffer may be connected in series in close proximity to each test pad 202 between the signal generator and the test pad. The buffer may comprise a narrow bandstop or notch filter (not shown) configured to filter mains frequency at around 50 Hz to 60 Hz which may be present on the line in embodiments where the test apparatus 200, in particular the signal generator 214, is powered by mains electricity.

The measurement circuit 216 may be configured to measure one or more electrical signals received at respective test electrodes 208 of the test apparatus 200 responsive to stimuli applied by the signal generator 214 at the test pads 202. The measurement circuit 216 may include one or more amplifiers, filters converters and/or other signal processing circuitry configured to precondition the signals received at the test electrodes 208 for processing by the processing unit 212.

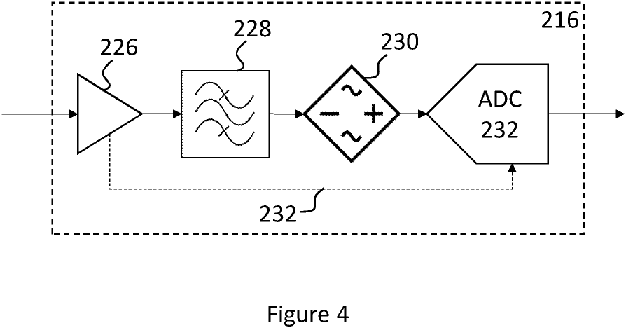

FIG. 4 is a schematic diagram of an example of the measurement circuit 216 according to some embodiments of the disclosure, specifically configured to determine the presence of a signal at a respective one of the test electrodes 208. Whilst in the embodiment shown a single signal chain is shown, the measurement circuit 216 may comprise additional signal chains in parallel; one for each test electrode 208. Alternatively, as described above, the measurement circuit 216 may be coupled to the test electrodes 208 via a multiplexer or n channel switch array (not shown). In which case the measurement circuit 216 may comprise a single signal chain as shown, the multiplexer being configured to address each test electrode to the measurement circuit 216 individually. The measurement circuit 216 comprises an amplifier 226, a bandpass filter 228, a rectifier 230 and an analogue-to-digital converter (ADC) 232. The selected signal from one of the test electrodes 208 is amplified by the amplifier 226 and passed to an optional bandpass filter 228. The bandpass filter 228 may be tuned to the frequency of oscillation of the signal applied by the signal generator 214 at the respective test pad 202, therefore reducing the risk of interference. The filtered AC signal is then passed to the rectifier 230 to be converted to a DC voltage which is then provided to the ADC 232 for conversion into the digital domain. The digital output signal is then provided to the I/O bus 222 of the processing unit 212 for further processing. This digital output provides an indication of whether a signal has been detected at a respective one of the test electrodes 208 and thus provides an indication as to whether a particular electrical path in the device 102 is functioning properly, or not.

In some embodiments, the measurement circuit 216 may comprise a signal path 232 configured to bypass the rectifier 230 and/or the bandpass filter 228. In doing so, the signal received from the amplifier 226 may be provided directly to the ADC 232 for conversion into the digital domain. This digital signal may in turn be provided to the processing unit 212 for analysis. In doing so, additional information concerning the connection under test may be ascertained. For example, impedance of the connection under test may be determined by performing a spectral analysis of the coupled signal received at the amplifier 226. Instead of a sine wave, a square wave may be applied by the signal generator to induce a step response in the signal received at the test electrodes 208. The signal may be applied at a range of frequencies and the signal received at the test electrode analysed in the frequency domain. A spectral analysis of the received signal may be used to determine an impedance of the connection under test and in doing so a determination may be made as to the characteristics of a defect in the device under test (such as a dry solder joint or the like). Such a determination may be made by comparing the measured impedance to an equivalent circuit model of the interface under test.

In some embodiments, the measurement circuit 216 may be external to or integrated within the processing unit 212. Communication between the measurement circuit 216 and/or the signal generator 214 on the one hand and the I/O bus 222 on the other may be wired or may be via a wireless link, such as over inductive coupling, WiFi (RTM), Bluetooth (RTM) or the like.

The test unit 204 may further comprise one or more input devices 222 and one or more output devices 224. Input devices 222 may include but are not limited to one or more of a keyboard, mouse, touchpad and touchscreen. Examples of output devices include displays, touchscreens, light indicators (LEDs), sound generators and haptic generators. Input and/or output devices 222, 224 may be configured to provide feedback (e.g. visual, auditory or haptic feedback) to a user related, for example, display test data generated by the test unit 204 in relation to the test being performed on the electronic device 102. To this end, one or more of the input devices 222 may also be an output device 224, e.g. a touchscreen. Input and output devices 222, 224 may also be wired or wirelessly connected to the processing unit 212. Input devices 222 may be configured to provide a user with control of test parameters used to test the device 102.

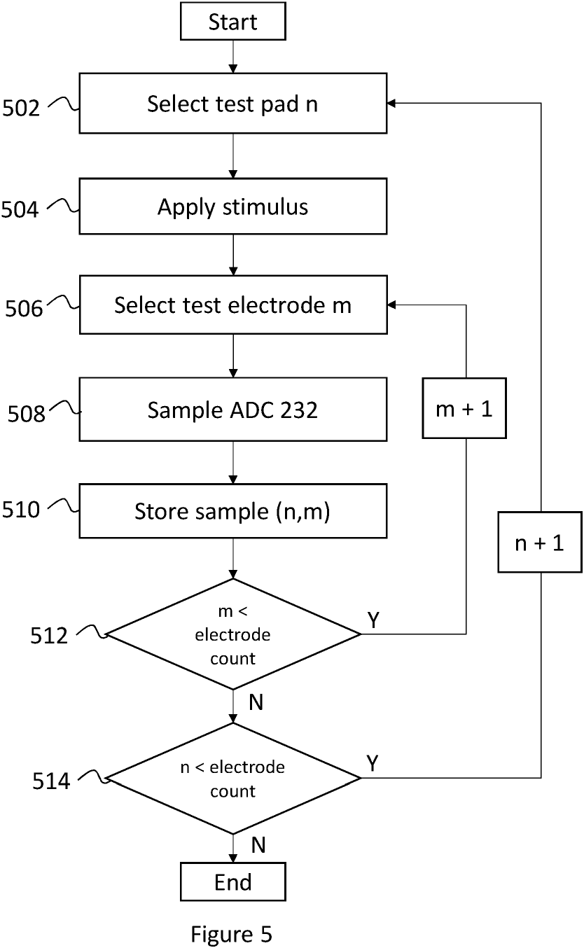

FIG. 5 is a flow diagram showing an exemplary process 500 performed by the test unit 204 of the test apparatus 200. Prior to implementing the process 500, a device to be tested, such as the electronic device 100 is placed on the test apparatus 200. Once secured, the camera 210 may capture an image of the device 100 as well as the barcode 120 which may be provided on the device 100 or device package 102. Captured images may be stored on the memory 220 provided in the processing unit 212. The test unit 204 may process the captured image of the device 100 to determine whether the device has any obvious visual defects. The test unit 204 may process the captured image of the barcode 120 to determine the type of device under test. This determination may be used during continuity (or other electrical characteristic) testing to determine how the device 100 should behave under test in response to stimuli applied at each test pad 202.

Once the device 100 has been identified and visual inspection has been performed, at step 502, the first test pad of n test pads 202 is selected and the processing unit 212 causes the signal generator 214 to apply a stimulus to that test pad at step 504. During application of the stimulus, at step 506, one of the m test electrodes 208 is selected and at step 508 the ADC 232 of the measurement circuit 216 samples the voltage at the selected test electrode m. The binary signal sampled from the ADC 232 is stored at step 510 in memory 222. At step 512, a determination is made as to whether the signal at all of the test electrodes 208 to be tested has been sampled and stored. If not, the process repeats steps 506 to 510 until all test electrodes 208 have been sampled. At step 514, a determination is made as to whether a stimulus has been applied to all test pads 202 being tested. If not, then steps 502 to 512 are repeated until a stimulus has been applied to all test pads 202 and respective signals at each of the test electrodes 208 have been sampled, and the process ends.

Using the above process 500, the continuity of each connection between the electrodes 104 of the electronic device 100 and the test electrodes 208 can be determined. In addition, it will be appreciated that other characteristics of signals received at the test electrodes 208 can be measured by the measurement circuit 216. On the basis of the measurements, additional characteristics of the device 100 may be determined including but not limited to impedance (discussed in more detail above).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A capacitive test apparatus for testing an electronic device contained within a package, the apparatus comprising:
a plurality of conductive pads in electrical communication with the processor, wherein the plurality of conductive pads are configured to align with electrodes of the electronic device during testing and capacitively couple electrical stimuli to the electrodes through the package;
processing circuitry configured to:
apply the electrical stimuli to the plurality of conductive pads;
receive one or more response signals induced by the electrical stimuli coupled through the package; and
determine one or more electrical characteristics of the electronic device based on the one or more response signals.

2. The apparatus of claim 1, wherein the package is at least partially comprised of a dielectric material.

3. The apparatus of claim 1, wherein the one or more characteristics comprises:
a) a continuity of an electrical connection in the electronic device;
b) an impedance of the electrical connection in the electronic device.

4. The apparatus of claim 1, wherein the processing circuitry is further configured to:
determine that the electronic device is operating properly based on the determining of the one or more characteristics.

5. The apparatus of claim 1, wherein the processing circuitry comprises a signal generator configured to generate the electrical stimuli.

6. The apparatus of claim 1, wherein the processing circuitry comprises one or more voltage buffers coupled to the one of the plurality of conductive pads.

7. The apparatus of claim 6, wherein the one or more voltage buffers each comprise a band-stop filter having a stop-band of between about 50 Hz and about 60 Hz.

8. The apparatus of claim 1, wherein the processing circuitry comprises a bandpass filter configured to filter one or more of the one or more response signals, the bandpass filter having a passband which spans the frequency of the electrical stimuli.

9. The apparatus of claim 1, wherein the processing circuitry comprises an analogue to digital converter configured to convert the one or more response signals into the digital domain.

10. The apparatus of claim 1, wherein the processing circuitry comprises a rectifier configured to rectify the one or more response signals.

11. The apparatus of claim 1, wherein the package is substantially transparent.

12. The apparatus of claim 1, further comprising a camera configured to capture an image of the electronic device and the package.

13. The apparatus of claim 12, wherein the processor is configured to process the image to determine a condition of the electronic device.

14. The apparatus of claim 12, wherein the processor is configured to read a barcode on the package captured in the image.

15. The apparatus of claim 1, further comprising a display for displaying the one or more electrical characteristics of the electronic device.

16. The apparatus of claim 1, further comprising one or more engagement elements configured to engage with features of the package so as to align the conductive pads with the respective electrodes of the electronic device during testing.

17. The apparatus of claim 1, further comprising one or more direct test electrodes configured to directly connect to a respective one or more contacts of the electronic device exposed through the package.

18. The apparatus of claim 17, wherein the one or more direct test electrodes each comprise a pogo pin configured to make direct connection with the one or more contacts during testing.

19. The apparatus of claim 17, wherein the one or more direct test electrodes directly connect to the one or more contacts via a self-healing membrane.

20. The apparatus of claim 1, further comprising the package, wherein the package comprises a plurality of contact regions each configured to receive a respective one of the plurality of test contacts to hold the respective one of the plurality of test contacts in place relative to the package and the plurality of conductive pads during testing.

21. The apparatus of claim 1, wherein the package is stackable.

22. A method for testing an electronic device housed within a package, the method comprising:
locating a plurality of conductive pads in proximity of respective electrodes of the electronic device;
capacitively coupling electrical stimuli from the conductive pads to the electrodes through the package;
receiving one or more response signals induced by the electrical stimuli coupled through the package; and
determining one or more electrical characteristics of the electronic device based on the one or more response signals.

23. The method of claim 22, wherein the package is at least partially comprised of a dielectric material.

24. The method of claim 22, wherein the one or more characteristics comprises:
a) a continuity of an electrical connection in the electronic device;
b) an impedance of the electrical connection in the electronic device.

25. The method of claim 22, further comprising:
determining that the electronic device is operating properly based on the determining of the one or more characteristics.

26. The method of claim 22, further comprising generating the electrical stimuli.

27. The method of claim 22, further comprising buffering the one or more response signals prior to determining the one or more electrical characteristics.

28. The method of claim 22, further comprising applying a band-stop filter to the one or more response signals, the band-stop filter having a stopband of between about 50 Hz and about 60 Hz.

29. The method of claim 22, further comprising applying a bandpass filter to one or more of the one or more response signals, the bandpass filter having a passband which spans the frequency of the electrical stimuli.

30. The method of claim 22, further comprising converting the one or more response signals into the digital domain.

31. The method of claim 22, further comprising rectifying the one or more response signals.

32. The method of claim 22, further comprising:
capturing an image of the electronic device and the package; and
processing the image to determine a condition of the electronic device and/or to read a barcode on the package captured in the image.

33. The method of claim 22, further comprising displaying the one or more electrical characteristics of the electronic device on a display.

34. The method of claim 22, wherein locating the plurality of conductive pads in proximity of respective electrodes of the electronic device comprises engaging one or more engagement elements associated with the conductive pads with features of the package so as to align the conductive pads with the respective electrodes of the electronic device during testing.

35. The method of claim 22, further comprising directly connecting one or more direct test electrodes to a respective one or more contacts of the electronic device exposed through the package.

36. The method of claim 35, wherein the one or more direct test electrodes each comprise a pogo pin configured to make direct connection with the one or more contacts during testing.

37. The method of claim 35, wherein the one or more direct test electrodes directly connect to the one or more contacts via a self-healing membrane.

38. The method of claim 22, further comprising locating each of the plurality of test contacts a respective one of a plurality of contact regions provided in the package, the plurality of contact regions configured to hold the respective one of the plurality of test contacts in place relative to the package and the plurality of conductive pads during testing.

39. (canceled)
